(19) United States
(12) Patent Application Publication
(10) Pub. No.: US 2005/0181049 A1
(43) Pub. Date: Aug. 18, 2005

(54) Composition and method for enhancing bioavailability

(76) Inventors: Liang C. Dong, Sunnyvale, CA (US); Crystal Pollock-Dove, Mountain View, CA (US); Jasmine Han, San Jose, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/984,401

(22) Filed: Nov. 9, 2004

Related U.S. Application Data
(60) Provisional application No. 60/523,421, filed on Nov. 19, 2003.

(51) Int. Cl. A61K 38/13; A61K 31/56; A61K 31/55; A61K 31/551; A61K 31/519

(52) U.S. Cl. 424/469; 514/220; 514/254.07; 514/253.08; 514/252.16; 514/11; 514/171; 514/649; 514/217; 514/259.41

(57) ABSTRACT

The present invention relates to compositions and methods for enhancing the bioavailability of beneficial agents with low water solubility.

### CROSS REFERENCE TO RELATED APPLICATIONS

This application claims benefit to U.S. Provisional Application No. 60/523,421, filed Nov. 19, 2003, the entirety of which is incorporated by reference herein.

### FIELD OF THE INVENTION

The present invention relates to compositions and methods for enhancing the bioavailability of beneficial agents with low water solubility.

### BACKGROUND OF THE INVENTION

Enhancing the dissolution and bioavailability of beneficial agents with low water solubility is of great interest in the art. Such compounds include all those that can be categorized as Class 2 by the United States Food and Drug Administration (FDA), which has issued a set of guidelines outlining the Biopharmaceutical Classification System (BCS). The BCS is a scientific framework for classifying drug substances based on their aqueous solubility and intestinal permeability. When combined with the dissolution of the drug product, the BCS takes into account three major factors that govern the rate and extent of drug absorption from IR solid assemblies: dissolution, solubility, and intestinal permeability. According to the BCS, drug substances are classified as follows: Class 1: High Solubility—High Permeability; Class 2: Low Solubility—High Permeability; Class 3: High Solubility—Low Permeability; and Class 4: Low Solubility—Low Permeability. Dissolution and/or solubilization in the gastro-intestinal tract and luminal transport of the dissolved molecules is the limiting step for absorption of Class 2 luminal transport of the dissolved molecules is the limiting step for absorption of Class 2 beneficial agents, and thus increasing dissolution rates is an important goal. Class 2 beneficial agents are a continuing challenge to administer because of problems associated with aggregation, precipitation, and difficulty preparing assemblies.

In the past, excellent results have been achieved with formulations that increase the solubility of Class 2 beneficial agents, including self-emulsifying liquid carrier formulations (“SEF”) that allow a beneficial agent to be more readily absorbed through a patient's gastrointestinal membranes and into the bloodstream as described in U.S. Pat. Nos. 6,419,952, 6,342,249, and 6,174,547. The disclosures of each of the foregoing documents are hereby incorporated herein by reference in their entireties.

However, it is always desirable to develop new methods to enhance the bioavailability of compounds with low water solubility, such as Class 2 beneficial agents. It has now been discovered that compositions and methods can be used to develop new assemblies for enhancing the bioavailability of Class 2 beneficial agents.

### SUMMARY OF THE INVENTION

Assemblies for delivering beneficial agents with low water solubility are described. The assemblies comprise porous-particle carriers contacted with mixtures comprising beneficial agents and water soluble polymers.

Methods of preparing an assembly for delivering beneficial agents with low water solubility are also described, the methods comprise providing porous-particle carriers, providing solutions comprising solvents, beneficial agents, and water soluble polymers, and applying the solutions to the carriers.

Similarly, methods of delivering beneficial agents with low water solubility to patients are described. Such methods comprise providing porous-particle carriers, providing solutions comprising solvents, beneficial agents, and water soluble polymers, applying the solutions to the carriers, and administering the loaded carriers to the patient.

### BRIEF DESCRIPTION OF THE DRAWINGS

FIG. 1 is a schematic of drug delivery according to one embodiment of the present invention.

### DETAILED DESCRIPTION

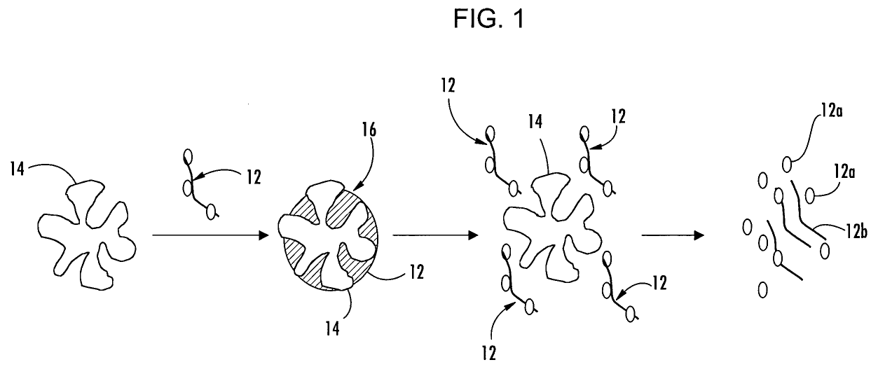

The present invention relates to compositions and methods for enhancing the bioavailability of beneficial agents with low water solubility. As shown in FIG. 1, a beneficial agent, in this embodiment a drug, is mixed with a polymer to form a drug/polymer complex 12. A porous carrier 14 is contacted by the drug/polymer complex 12 to create an assembly 16. If desired, such assemblies could be readily incorporated into a conventional beneficial agent delivery platform (not depicted). When the assembly 16 is placed in an aqueous medium, such as upon administration to a patient, the drug/polymer complex 12 disassociates from the carrier 14. Likewise, the drug/polymer complex 12 itself dissociates to its component drug 12a and polymer 12b moieties, thereby making the drug available for absorption.

In one embodiment, the present invention includes an assembly for delivering a beneficial agent with low water solubility, comprising a porous-particle carrier contacted with a mixture comprising the beneficial agent and a water soluble polymer.

Porous-particles that are useful are characterized by high compressibility or tensile strength, high porosity, and low friability. The porous-particle carrier is selected from magnesium aluminometasilicate, anhydrous dibasic calcium phosphate, microcrystalline cellulose, cross linked sodium carboxymethyl cellulose, soy bean hull fiber, and agglomerated silicon dioxide.

Magnesium aluminometasilicate (Al₂O₃.MgO.1.7SiO₂.xH₂O) is available from Fuji Chemical Industry Co., Ltd, Japan, under the tradename NEUSILIN. Magnesium aluminometasilicate may be represented by the general formula Al₂O₃.MgO.xSiO₂ nH₂O, wherein x is in a range of about 1.5 to about 2, and n satisfies the relationship 0≦n≦10.

Anhydrous dibasic calcium phosphate (CaHPO₄) is available from Fuji Chemical Industry Co., Ltd, Japan, under the tradename FUJICALIN. A particularly suitable porous-particle is exemplified by the particular form of calcium hydrogen phosphate described in U.S. Pat. No. 5,486,365, which is incorporated herein by reference in its entirety. As described therein, calcium hydrogen phosphate is prepared by a process yielding a scale-like calcium hydrogen phosphate that can be represented by the formula CaHPO₄ mH₂O wherein m satisfies the expression 0≦m≦2.0.

Microcrystalline cellulose is available under the tradename AVICEL from FMC BioPolymer, Philadelphia, Pa., USA, and under the tradename ELCEMA from Degussa AG, Germany.

Cross linked sodium carboxymethyl cellulose is available under the tradename AC-DI-SOL from FMC BioPolymer, Philadelphia, Pa., USA.

Soy bean hull fiber is available under the tradename FL-1 SOY FIBER from Fibred Group, Cumberland, Md., USA.

Agglomerated silicon dioxide is available under the tradename CAB-O-SIL from Cabot Corporation, Boston, Mass., USA, and is available under the tradename AEROSIL from Degussa AG, Germany.

Preferably, the porous-particle carrier is magnesium aluminometasilicate or anhydrous dibasic calcium phosphate, and more preferably the porous-particle carrier is magnesium aluminometasilicate.

Preferably, the porous-particle carrier is present in a range from about 20% to about 99% by weight of the assembly. More preferably, the porous-particle carrier is present in a range from about 40% to about 99% by weight of the assembly. In one embodiment, the porous-particle carrier is present in a range from about 40% to about 60% by weight of the assembly. In another embodiment, the porous-particle carrier is present in a range from about 50% to about 99% by weight of the assembly. In yet another embodiment, the porous-particle carrier is present in a range from about 60% to about 80% by weight of the assembly.

Beneficial agents used in the present invention include all those compounds known to have an effect on humans or animals that also have low water solubility. Such compounds include all those that can be categorized as Class 2 under the Biopharmaceutical Classification System (BCS) set out by the United States Food and Drug Administration (FDA). Determining which BCS Class a drug bellows in is a matter of routine experimentation, well known to those skilled in the art.

Exemplary beneficial agents that can be delivered by the osmotic system of this invention include prochlorperazine edisylate, ferrous sulfate, aminocaproic acid, potassium chloride, mecamylamine hydrochloride, procainamide hydrochloride, amphetamine sulfate, benzphetamine hydrochloride, isoprotemol sulfate, methamphetamine hydrochloride, phenmetrazine hydrochloride, bethanechol chloride, metacholine chloride, pilocarpine hydrochloride, atropine sulfate, methascopolamine bromide, isopropamide iodide, tridihexethyl chloride, phenformin hydrochloride, methylphenidate hydrochloride, oxprenolol hydrochloride, metroprolol tartrate, cimetidine hydrochloride, diphenidol, meclizine hydrochloride, prochlorperazine maleate, phenoxybenzamine, thiethylperazine, maleate, anisindone, diphenadione erythrityl teranitrate, digoxin, isofurophate, reserpine, acetazolamide, methazolamide, bendroflumethiazide, chlorpropamide, tolazamide, chlormadinone acetate, phenaglycodol, allopurinol, aluminum aspirin, methotrexate, acetyl sulfisoxazole, erythromycin, progestins, estrogenic progrestational, corticosteroids, hydrocortisone, hydrocorticosterone acetate, cortisone acetate, triamcinolone, methyltesterone, 17 β-estradiol, ethinyl estradiol, ethinyl estradiol 3-methyl ether, prednisolone, 17-hydroxyprogesterone acetate, 19-nor-progesterone, norgestrel orethindone, norethiderone, progesterone, norgestrone, norethynodrel, aspirin, indomethacin, naproxen, fenoprofen, sulindac, diclofenac, indoprofen, nitroglycerin, propranolol, metroprolol, sodium valproate, valproic acid, taxanes such as paclitaxel, camptothecins such as 9-aminocamptothecin, oxprenolol, timolol, atenolol, alprenolol, cimetidine, clonidine, imipramine, levodopa, chloropropmazine, resperine, methyldopa, dihydroxyphenylalanine, pivaloyloxyethyl ester of a-methyldopa hydrochloride, theophylline, calcium gluconate ferrous lactate, ketoprofen, ibuprofen, cephalexin, haloperiodol, zomepirac, vincamine, diazepam, phenoxybenzamine, nifedipine, diltiazen, verapamil, lisinopril, captopril, ramipril, fosimopril, benazepril, libenzapril, cilazapril cilazaprilat, perindopril, zofenopril, enalapril, indalapril, qumapril, megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, budesnonide, progesterone, megestrol acetate, topiramate, naproxen, flurbiprofen, ketoprofen, desipramine, diclofenac, itraconazole, piroxicam, carbamazepine, phenytoin, and verapamil, indinavir sulfate, lamivudine, stavudine, nelfinavir mesylate, a combination of lamivudine and zidovudine, saquinavir mesylate, ritonavir, zidovudine, didanosine, nevirapine, ganciclovir, zalcitabine, fluoexetine hydrochloride, sertraline hydrochloride, paroxetine hydrochloride, bupropion hydrochloride, nefazodone hydrochloride, mirtazpine, auroix, mianserin hydrochloride, zanamivir, olanzapine, risperidone, quetiapine fumurate, buspirone hydrochloride, alprazolam, lorazepam, leotan, clorazepate dipotassium, clozapine, sulpiride, amisulpride, methylphenidate hydrochloride, and pemoline.

Beneficial agents having low water solubility, e.g., less than 50 micrograms/ml, are useful with the present invention. Beneficial agents include megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, budesnonide, progesterone, megestrol acetate, topiramate, naproxen, flurbiprofen, ketoprofen, desipramine, diclofenac, itraconazole, piroxicam, carbamazepine, phenytoin, verapamil, indinavir sulfate, lamivudine, stavudine, nelfinavir mesylate, a combination of lamivudine and zidovudine, saquinavir mesylate, ritonavir, zidovudine, didanosine, nevirapine, ganciclovir, zalcitabine, fluoexetine hydrochloride, sertraline hydrochloride, paroxetine hydrochloride, bupropion hydrochloride, nefazodone hydrochloride, mirtazpine, auroix, mianserin hydrochloride, zanamivir, olanzapine, risperidone, quetiapine fumurate, buspirone hydrochloride, alprazolam, lorazepam, leotan, clorazepate dipotassium, clozapine, sulpiride, amisulpride, methylphenidate hydrochloride, and pemoline.

Preferably, the beneficial agents include megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, budesnonide, progesterone, megestrol acetate, topiramate, naproxen, flurbiprofen, ketoprofen, desipramine, diclofenac, itraconazole, piroxicam, carbamazepine, phenytoin, and verapamil. More preferably, such compounds include megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, and budesnonide.

Preferably, beneficial agent is present in a range from about 1% to about 60% by weight of the assembly, and more preferably the beneficial agent is present in a range from about 40% to about 60% by weight of the assembly.

Without restriction by the foregoing, the beneficial agent is preferably present in a range from about 0.1 mg to about 500 mg, and more preferably the beneficial agent is present in a range from about 20 mg to about 250 mg.

Other beneficial agents known to the art are incorporated as well, as described in Pharmaceutical Sciences, 14th Ed., 1979, Mack Publishing Co., Easton, Pa.; The Beneficial Agent, The Nurse, The Patient, Including Current Beneficial Agent Handbook, 1976, Saunder Company, Philadelphia, Pa.; Medical Chemistry, 3rd Ed., Vol. 1 and 2, Wiley-Interscience, New York; and, Physician's Desk Reference, 55nd Ed., 1998, Medical Economics Co., New Jersey. It is understood that the beneficial agent may be in various forms such as unchanged molecules, molecular complexes, pharmacologically acceptable salts such as hydrochloride, hydrobromide, sulfate, laurate, palmitate, phosphate, nitrite, nitrate, borate, acetate, maleate, tartrate, oleate, salicylate, and the like. For acidic beneficial agents, salts of metals, amines, or organic cations, for example quaternary ammonium can be used. Derivatives of beneficial agents, such as bases, ester, ether and amide can be used.

The polymer is ethyl(hydroxyethyl)cellulose available from Berol Nobel, Sweden, hydroxypropyl methylcellulose available from The Dow Chemical Company, USA, under the tradename METHOCEL, hydroxyethyl cellulose modified with hydrophobic groups, such as CELLULOSE HEC SPLATTER GUARD 100 available from The Dow Chemical Company, USA, anionic copolymers based on methacrylic acid and methyl methacrylate, for example having a ratio of free carboxyl groups to methyl-esterified carboxyl groups of 1:>3 (i.e., about 1:1 or about 1:2) with a mean molecular weight of 135000, available under the tradename EUDRAGIT from Degussa AG, Germany (Röhm subsidiary), or any enteric polymer.

Preferred polymers include more hydrophobic hydroxypropyl methylcellulose, such as is available under the tradenames METHOCEL E, METHOCEL J, and METHOCEL HB all from The Dow Chemical Company, USA, and methacrylic acid copolymers, such as is available under the tradename EUDRAGIT L and EUDRAGIT S both from Degussa AG, Germany. The most preferred polymer is hydroxypropyl methylcellulose.

Preferably, the water soluble polymer is present in a range from about 1% to about 50% by weight of the assembly, and more preferably the water soluble polymer is present in a range from about 10% to about 30% by weight of the assembly.

In another embodiment of the present invention, a method of preparing an assembly for delivering a beneficial agent with low water solubility is described, comprising providing a porous-particle carrier, providing a solution comprising a solvent, the beneficial agent, and a water soluble polymer; and applying the solution to the carrier.

The solution may be applied by contacting the carrier with the solution by any conventional means, including spraying.

The solvent is water, acetone, ethanol, methanol, dimethyl sulfoxide (“DMSO”), methylene chloride, and mixtures thereof. In one embodiment, the solvent is ethanol and water. In another embodiment, the solvent is ethanol and DMSO. In yet another embodiment, the solvent is DMSO.

Porous-particles that are useful are characterized by high compressibility or tensile strength, high porosity, and low friability. The porous-particle carrier is selected from magnesium aluminometasilicate, anhydrous dibasic calcium phosphate, microcrystalline cellulose, cross linked sodium carboxymethyl cellulose, soy bean hull fiber, and agglomerated silicon dioxide.

Magnesium aluminometasilicate (Al₂O₃.MgO.1.7SiO₂.xH₂O) is available from Fuji Chemical Industry Co., Ltd, Japan, under the tradename NEUSILIN. Magnesium aluminometasilicate may be represented by the general formula Al₂O₃.MgO.xSiO₂ nH₂O, wherein x is in a range of about 1.5 to about 2, and n satisfies the relationship 0≦n≦10.

Anhydrous dibasic calcium phosphate (CaHPO₄) is available from Fuji Chemical Industry Co., Ltd, Japan, under the tradename FUJICALIN. A particularly suitable porous-particle is exemplified by the particular form of calcium hydrogen phosphate described in U.S. Pat. No. 5,486,365, which is incorporated herein by reference in its entirety. As described therein, calcium hydrogen phosphate is prepared by a process yielding a scale-like calcium hydrogen phosphate that can be represented by the formula CaHPO₄ mH₂O wherein m satisfies the expression 0≦m≦2.0.

Microcrystalline cellulose is available under the tradename AVICEL from FMC BioPolymer, Philadelphia, Pa., USA, and under the tradename ELCEMA from Degussa AG, Germany.

Cross linked sodium carboxymethyl cellulose is available under the tradename AC-DI-SOL from FMC BioPolymer, Philadelphia, Pa., USA.

Soy bean hull fiber is available under the tradename FL-1 SOY FIBER from Fibred Group, Cumberland, Md., USA.

Agglomerated silicon dioxide is available under the tradename CAB-O-SIL from Cabot Corporation, Boston, Mass., USA, and is available under the tradename AEROSIL from Degussa AG, Germany.

Preferably, the porous-particle carrier is magnesium aluminometasilicate or anhydrous dibasic calcium phosphate, and more preferably the porous-particle carrier is magnesium aluminometasilicate.

Preferably, the porous-particle carrier is present in a range from about 20% to about 99% by weight of the assembly. More preferably, the porous-particle carrier is present in a range from about 40% to about 99% by weight of the assembly. In one embodiment, the porous-particle carrier is present in a range from about 40% to about 60% by weight of the assembly. In another embodiment, the porous-particle carrier is present in a range from about 50% to about 99% by weight of the assembly. In yet another embodiment, the porous-particle carrier is present in a range from about 60% to about 80% by weight of the assembly.

Beneficial agents used in the present invention include all those compounds known to have an effect on humans or animals that also have low water solubility. Such compounds include all those that can be categorized as Class 2 under the Biopharmaceutical Classification System (BCS) set out by the United States Food and Drug Administration (FDA). Determining which BCS Class a drug bellows in is a matter of routine experimentation, well known to those skilled in the art.

Exemplary beneficial agents that can be delivered by the osmotic system of this invention include prochlorperazine edisylate, ferrous sulfate, aminocaproic acid, potassium chloride, mecamylamine hydrochloride, procainamide hydrochloride, amphetamine sulfate, benzphetamine hydrochloride, isoproternol sulfate, methamphetamine hydrochloride, phenmetrazine hydrochloride, bethanechol chloride, metacholine chloride, pilocarpine hydrochloride, atropine sulfate, methascopolamine bromide, isopropamide iodide, tridihexethyl chloride, phenformin hydrochloride, methylphenidate hydrochloride, oxprenolol hydrochloride, metroprolol tartrate, cimetidine hydrochloride, diphenidol, meclizine hydrochloride, prochlorperazine maleate, phenoxybenzamine, thiethylperazine, maleate, anisindone, diphenadione erythrityl teranitrate, digoxin, isofurophate, reserpine, acetazolamide, methazolamide, bendroflumethiazide, chlorpropamide, tolazamide, chlormadinone acetate, phenaglycodol, allopurinol, aluminum aspirin, methotrexate, acetyl sulfisoxazole, erythromycin, progestins, estrogenic progrestational, corticosteroids, hydrocortisone, hydrocorticosterone acetate, cortisone acetate, triamcinolone, methyltesterone, 17 β-estradiol, ethinyl estradiol, ethinyl estradiol 3-methyl ether, prednisolone, 17 -hydroxyprogesterone acetate, 19 -nor-progesterone, norgestrel orethindone, norethiderone, progesterone, norgestrone, norethynodrel, aspirin, indomethacin, naproxen, fenoprofen, sulindac, diclofenac, indoprofen, nitroglycerin, propranolol, metroprolol, sodium valproate, valproic acid, taxanes such as paclitaxel, camptothecins such as 9-aminocamptothecin, oxprenolol, timolol, atenolol, alprenolol, cimetidine, clonidine, imipramine, levodopa, chloropropmazine, resperine, methyldopa, dihydroxyphenylalanine, pivaloyloxyethyl ester of a-methyldopa hydrochloride, theophylline, calcium gluconate ferrous lactate, ketoprofen, ibuprofen, cephalexin, haloperiodol, zomepirac, vincamine, diazepam, phenoxybenzamine, nifedipine, diltiazen, verapamil, lisinopril, captopril, ramipril, fosimopril, benazepril, libenzapril, cilazapril cilazaprilat, perindopril, zofenopril, enalapril, indalapril, qumapril, megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, budesnonide, progesterone, megestrol acetate, topiramate, naproxen, flurbiprofen, ketoprofen, desipramine, diclofenac, itraconazole, piroxicam, carbamazepine, phenytoin, and verapamil, indinavir sulfate, lamivudine, stavudine, nelfinavir mesylate, a combination of lamivudine and zidovudine, saquinavir mesylate, ritonavir, zidovudine, didanosine, nevirapine, ganciclovir, zalcitabine, fluoexetine hydrochloride, sertraline hydrochloride, paroxetine hydrochloride, bupropion hydrochloride, nefazodone hydrochloride, mirtazpine, auroix, mianserin hydrochloride, zanamivir, olanzapine, risperidone, quetiapine fumurate, buspirone hydrochloride, alprazolam, lorazepam, leotan, clorazepate dipotassium, clozapine, sulpiride, amisulpride, methylphenidate hydrochloride, and pemoline.

Beneficial agents having low water solubility, e.g., less than 50 micrograms/ml, are useful with the present invention. Beneficial agents include megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, budesnonide, progesterone, megestrol acetate, topiramate, naproxen, flurbiprofen, ketoprofen, desipramine, diclofenac, itraconazole, piroxicam, carbamazepine, phenytoin, verapamil, indinavir sulfate, lamivudine, stavudine, nelfinavir mesylate, a combination of lamivudine and zidovudine, saquinavir mesylate, ritonavir, zidovudine, didanosine, nevirapine, ganciclovir, zalcitabine, fluoexetine hydrochloride, sertraline hydrochloride, paroxetine hydrochloride, bupropion hydrochloride, nefazodone hydrochloride, mirtazpine, auroix, mianserin hydrochloride, zanamivir, olanzapine, risperidone, quetiapine fumurate, buspirone hydrochloride, alprazolam, lorazepam, leotan, clorazepate dipotassium, clozapine, sulpiride, amisulpride, methylphenidate hydrochloride, and pemoline.

Preferably, the beneficial agents include megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, budesnonide, progesterone, megestrol acetate, topiramate, naproxen, flurbiprofen, ketoprofen, desipramine, diclofenac, itraconazole, piroxicam, carbamazepine, phenytoin, and verapamil. More preferably, such compounds include megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, and budesnonide.

Preferably, beneficial agent is present in a range from about 1% to about 60% by weight of the assembly, and more preferably the beneficial agent is present in a range from about 40% to about 60% by weight of the assembly.

Without restriction by the foregoing, the beneficial agent is preferably present in a range from about 0.1 mg to about 500 mg, and more preferably the beneficial agent is present in a range from about 20 mg to about 250 mg.

Other beneficial agents known to the art are incorporated as well, as described in Pharmaceutical Sciences, 14th Ed., 1979, Mack Publishing Co., Easton, Pa.; The Beneficial Agent, The Nurse, The Patient, Including Current Beneficial Agent Handbook, 1976, Saunder Company, Philadelphia, Pa.; Medical Chemistry, 3rd Ed., Vol. 1 and 2, Wiley-Interscience, New York; and, Physician's Desk Reference, 55nd Ed., 1998, Medical Economics Co., New Jersey. It is understood that the beneficial agent may be in various forms such as unchanged molecules, molecular complexes, pharmacologically acceptable salts such as hydrochloride, hydrobromide, sulfate, laurate, palmitate, phosphate, nitrite, nitrate, borate, acetate, maleate, tartrate, oleate, salicylate, and the like. For acidic beneficial agents, salts of metals, amines, or organic cations, for example quaternary ammonium can be used. Derivatives of beneficial agents, such as bases, ester, ether and amide can be used.

The polymer is ethyl(hydroxyethyl)cellulose available from Berol Nobel, Sweden, hydroxypropyl methylcellulose available from The Dow Chemical Company, USA, under the tradename METHOCEL, hydroxyethyl cellulose modified with hydrophobic groups, such as CELLULOSE HEC SPLATTER GUARD 100 available from The Dow Chemical Company, USA, anionic copolymers based on methacrylic acid and methyl methacrylate, for example having a ratio of free carboxyl groups to methyl-esterified carboxyl groups of 1:>3 (i.e., about 1:1 or about 1:2) with a mean molecular weight of 135000, available under the tradename EUDRAGIT from Degussa AG, Germany (Röhm subsidiary), or any enteric polymer.

Preferred polymers include more hydrophobic hydroxypropyl methylcellulose, such as is available under the tradenames METHOCEL E, METHOCEL J, and METHOCEL HB all from The Dow Chemical Company, USA, and methacrylic acid copolymers, such as is available under the tradename EUDRAGIT L and EUDRAGIT S both from Degussa AG, Germany. The most preferred polymer is hydroxypropyl methylcellulose.

Preferably, the water soluble polymer is present in a range from about 1% to about 50% by weight of the assembly, and more preferably the water soluble polymer is present in a range from about 10% to about 30% by weight of the assembly.

In yet another embodiment of the present invention, a method of delivering a beneficial agent with low water solubility to a patient is described, comprising providing a porous-particle carrier, providing a solution comprising a solvent, the beneficial agent, and a water soluble polymer, applying the solution to the carrier; and administering the loaded carrier to the patient.

The solution may be applied by contacting the carrier with the solution by any conventional means, including spraying.

The administration may be by any conventional means, including via a delivery system. In terms of beneficial agent delivery systems, excellent results have been achieved with ALZA's OROS™ system, which uses osmosis technology to allow a beneficial agent to be more readily absorbed through a patient's gastrointestinal membranes and into the bloodstream. A beneficial agent layer and an osmotic engine are encased in a hard capsule surrounded by a rate-controlling semipermeable membrane, as described in U.S. Pat. No. 5,770,227, the disclosure of which is hereby incorporated herein by reference in its entirety. In summary, a barrier layer, composed of an inert substance, separates the beneficial agent layer from the osmotic engine, preventing the beneficial agent from reacting with the osmotic engine. A delivery orifice, laser drilled in the membrane at the end opposite from the osmotic engine, provides an outlet for the beneficial agent. Preferred delivery systems include ALZA's OROS™ PUSH-STICK™ beneficial agent delivery system (designed to deliver insoluble drugs requiring high loading, with an optimal delayed, patterned, or pulsatile release profile), ALZA's OROS™ PUSH-PULL™ beneficial agent delivery system (designed to deliver drugs ranging from low to high water solubility), and a matrix tablet beneficial agent delivery system.

Generally, beneficial agents may be administered to a patient by any known method in dosages ranging from about 0.001 to about 1.0 mmoles per kg body weight (and all combinations and subcombinations of dosage ranges and specific dosages therein). The useful dosage to be administered and the particular mode of administration will vary depending upon such factors as age, weight, and problem to be treated, as well as the particular beneficial agent used, as will be readily apparent to those skilled in the art. Typically, dosage is administered at lower levels and increased until the desirable diagnostic effect is achieved.

The solvent is water, acetone, ethanol, methanol, dimethyl sulfoxide (“DMSO”), methylene chloride, and mixtures thereof. In one embodiment, the solvent is ethanol and water. In another embodiment, the solvent is ethanol and DMSO. In yet another embodiment, the solvent is DMSO.

Porous-particles that are useful are characterized by high compressibility or tensile strength, high porosity, and low friability. The porous-particle carrier is selected from magnesium aluminometasilicate, anhydrous dibasic calcium phosphate, microcrystalline cellulose, cross linked sodium carboxymethyl cellulose, soy bean hull fiber, and agglomerated silicon dioxide.

Magnesium aluminometasilicate (Al₂O₃.MgO.1.7SiO₂.xH₂O) is available from Fuji Chemical Industry Co., Ltd, Japan, under the tradename NEUSILIN. Magnesium aluminometasilicate may be represented by the general formula Al₂O₃.MgO.xSiO₂ nH₂O, wherein x is in a range of about 1.5 to about 2, and n satisfies the relationship 0≦n≦10.

Anhydrous dibasic calcium phosphate (CaHPO₄) is available from Fuji Chemical Industry Co., Ltd, Japan, under the tradename FUJICALIN. A particularly suitable porous-particle is exemplified by the particular form of calcium hydrogen phosphate described in U.S. Pat. No. 5,486,365, which is incorporated herein by reference in its entirety. As described therein, calcium hydrogen phosphate is prepared by a process yielding a scale-like calcium hydrogen phosphate that can be represented by the formula CaHPO₄ mH₂O wherein m satisfies the expression 0≦m≦2.0.

Microcrystalline cellulose is available under the tradename AVICEL from FMC BioPolymer, Philadelphia, Pa., USA, and under the tradename ELCEMA from Degussa AG, Germany.

Cross linked sodium carboxymethyl cellulose is available under the tradename AC-DI-SOL from FMC BioPolymer, Philadelphia, Pa., USA.

Soy bean hull fiber is available under the tradename FL-1 SOY FIBER from Fibred Group, Cumberland, Md., USA.

Agglomerated silicon dioxide is available under the tradename CAB-O-SIL from Cabot Corporation, Boston, Ma., USA, and is available under the tradename AEROSIL from Degussa AG, Germany.

Preferably, the porous-particle carrier is magnesium aluminometasilicate or anhydrous dibasic calcium phosphate, and more preferably the porous-particle carrier is magnesium aluminometasilicate.

Preferably, the porous-particle carrier is present in a range from about 20% to about 99% by weight of the assembly. More preferably, the porous-particle carrier is present in a range from about 40% to about 99% by weight of the assembly. In one embodiment, the porous-particle carrier is present in a range from about 40% to about 60% by weight of the assembly. In another embodiment, the porous-particle carrier is present in a range from about 50% to about 99% by weight of the assembly. In yet another embodiment, the porous-particle carrier is present in a range from about 60% to about 80% by weight of the assembly.

Beneficial agents used in the present invention include all those compounds known to have an effect on humans or animals that also have low water solubility. Such compounds include all those that can be categorized as Class 2 under the Biopharmaceutical Classification System (BCS) set out by the United States Food and Drug Administration (FDA). Determining which BCS Class a drug bellows in is a matter of routine experimentation, well known to those skilled in the art.

Exemplary beneficial agents that can be delivered by the osmotic system of this invention include prochlorperazine edisylate, ferrous sulfate, aminocaproic acid, potassium chloride, mecamylamine hydrochloride, procainamide hydrochloride, amphetamine sulfate, benzphetamine hydrochloride, isoprotemol sulfate, methamphetamine hydrochloride, phenmetrazine hydrochloride, bethanechol chloride, metacholine chloride, pilocarpine hydrochloride, atropine sulfate, methascopolamine bromide, isopropamide iodide, tridihexethyl chloride, phenformin hydrochloride, methylphenidate hydrochloride, oxprenolol hydrochloride, metroprolol tartrate, cimetidine hydrochloride, diphenidol, meclizine hydrochloride, prochlorperazine maleate, phenoxybenzamine, thiethylperazine, maleate, anisindone, diphenadione erythrityl teranitrate, digoxin, isofurophate, reserpine, acetazolamide, methazolamide, bendroflumethiazide, chlorpropamide, tolazamide, chlormadinone acetate, phenaglycodol, allopurinol, aluminum aspirin, methotrexate, acetyl sulfisoxazole, erythromycin, progestins, estrogenic progrestational, corticosteroids, hydrocortisone, hydrocorticosterone acetate, cortisone acetate, triamcinolone, methyltesterone, 17 β-estradiol, ethinyl estradiol, ethinyl estradiol 3-methyl ether, prednisolone, 17 -hydroxyprogesterone acetate, 19 -nor-progesterone, norgestrel orethindone, norethiderone, progesterone, norgestrone, norethynodrel, aspirin, indomethacin, naproxen, fenoprofen, sulindac, diclofenac, indoprofen, nitroglycerin, propranolol, metroprolol, sodium valproate, valproic acid, taxanes such as paclitaxel, camptothecins such as 9-aminocamptothecin, oxprenolol, timolol, atenolol, alprenolol, cimetidine, clonidine, imipramine, levodopa, chloropropmazine, resperine, methyldopa, dihydroxyphenylalanine, pivaloyloxyethyl ester of a-methyldopa hydrochloride, theophylline, calcium gluconate ferrous lactate, ketoprofen, ibuprofen, cephalexin, haloperiodol, zomepirac, vincamine, diazepam, phenoxybenzamine, nifedipine, diltiazen, verapamil, lisinopril, captopril, ramipril, fosimopril, benazepril, libenzapril, cilazapril cilazaprilat, perindopril, zofenopril, enalapril, indalapril, qumapril, megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, budesnonide, progesterone, megestrol acetate, topiramate, naproxen, flurbiprofen, ketoprofen, desipramine, diclofenac, itraconazole, piroxicam, carbamazepine, phenytoin, and verapamil, indinavir sulfate, lamivudine, stavudine, nelfinavir mesylate, a combination of lamivudine and zidovudine, saquinavir mesylate, ritonavir, zidovudine, didanosine, nevirapine, ganciclovir, zalcitabine, fluoexetine hydrochloride, sertraline hydrochloride, paroxetine hydrochloride, bupropion hydrochloride, nefazodone hydrochloride, mirtazpine, auroix, mianserin hydrochloride, zanamivir, olanzapine, risperidone, quetiapine fumurate, buspirone hydrochloride, alprazolam, lorazepam, leotan, clorazepate dipotassium, clozapine, sulpiride, amisulpride, methylphenidate hydrochloride, and pemoline.

Beneficial agents having low water solubility, e.g., less than 50 micrograms/ml, are useful with the present invention. Beneficial agents include megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, budesnonide, progesterone, megestrol acetate, topiramate, naproxen, flurbiprofen, ketoprofen, desipramine, diclofenac, itraconazole, piroxicam, carbamazepine, phenytoin, verapamil, indinavir sulfate, lamivudine, stavudine, nelfinavir mesylate, a combination of lamivudine and zidovudine, saquinavir mesylate, ritonavir, zidovudine, didanosine, nevirapine, ganciclovir, zalcitabine, fluoexetine hydrochloride, sertraline hydrochloride, paroxetine hydrochloride, bupropion hydrochloride, nefazodone hydrochloride, mirtazpine, auroix, mianserin hydrochloride, zanamivir, olanzapine, risperidone, quetiapine fumurate, buspirone hydrochloride, alprazolam, lorazepam, leotan, clorazepate dipotassium, clozapine, sulpiride, amisulpride, methylphenidate hydrochloride, and pemoline.

Preferably, the beneficial agents include megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, budesnonide, progesterone, megestrol acetate, topiramate, naproxen, flurbiprofen, ketoprofen, desipramine, diclofenac, itraconazole, piroxicam, carbamazepine, phenytoin, and verapamil. More preferably, such compounds include megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, and budesnonide.

Preferably, beneficial agent is present in a range from about 1% to about 60% by weight of the assembly, and more preferably the beneficial agent is present in a range from about 40% to about 60% by weight of the assembly.

Without restriction by the foregoing, the beneficial agent is preferably present in a range from about 0.1 mg to about 500 mg, and more preferably the beneficial agent is present in a range from about 20 mg to about 250 mg.

Other beneficial agents known to the art are incorporated as well, as described in Pharmaceutical Sciences, 14th Ed., 1979, Mack Publishing Co., Easton, Pa.; The Beneficial Agent, The Nurse, The Patient, Including Current Beneficial Agent Handbook, 1976, Saunder Company, Philadelphia, Pa.; Medical Chemistry, 3rd Ed., Vol. 1 and 2, Wiley-Interscience, New York; and, Physician's Desk Reference, 55nd Ed., 1998, Medical Economics Co., New Jersey. It is understood that the beneficial agent may be in various forms such as unchanged molecules, molecular complexes, pharmacologically acceptable salts such as hydrochloride, hydrobromide, sulfate, laurate, palmitate, phosphate, nitrite, nitrate, borate, acetate, maleate, tartrate, oleate, salicylate, and the like. For acidic beneficial agents, salts of metals, amines, or organic cations, for example quaternary ammonium can be used. Derivatives of beneficial agents, such as bases, ester, ether and amide can be used.

The polymer is ethyl(hydroxyethyl)cellulose available from Berol Nobel, Sweden, hydroxypropyl methylcellulose available from The Dow Chemical Company, USA, under the tradename METHOCEL, hydroxyethyl cellulose modified with hydrophobic groups, such as CELLULOSE HEC SPLATTER GUARD 100 available from The Dow Chemical Company, USA, anionic copolymers based on methacrylic acid and methyl methacrylate, for example having a ratio of free carboxyl groups to methyl-esterified carboxyl groups of 1:>3 (i.e., about 1:1 or about 1:2) with a mean molecular weight of 135000, available under the tradename EUDRAGIT from Degussa AG, Germany (Röhm subsidiary), or any enteric polymer.

Preferred polymers include more hydrophobic hydroxypropyl methylcellulose, such as is available under the tradenames METHOCEL E, METHOCEL J, and METHOCEL HB all from The Dow Chemical Company, USA, and methacrylic acid copolymers, such as is available under the tradename EUDRAGIT L and EUDRAGIT S both from Degussa AG, Germany. The most preferred polymer is hydroxypropyl methylcellulose.

Preferably, the water soluble polymer is present in a range from about 1% to about 50% by weight of the assembly, and more preferably the water soluble polymer is present in a range from about 10% to about 30% by weight of the assembly.

The present invention is further described in the following examples.

### EXAMPLES

### Example 1

Magnesium aluminometasilicate is loaded by an iterative spraying/drying process in a fluid bed granulator using a 50/50 wt % solution of itraconazol and hydroxypropyl methylcellulose (“HPMC”) available under the tradename METHOCEL E5 in DMSO with 6% solids. The solution is rapidly sprayed onto the fluidized porous particles (magnesium aluminometasilicate), conservatively only loading 75% of the pores' absorbing capacity. Then the spraying is stopped while heating and fluidizing continues, allowing the solvent to evaporate leaving the drug/polymer solids behind trapped inside the pores. The process is repeated, scaling down the amount of solution applied each cycle proportional to the amount of the remaining percentage of unfilled pores. The pores will be 75% filled with drug/polymer solids after 10 iterations. Assuming 50% porosity, the final composition of the assembly is carrier/drug/polymer in a ratio of about 72:14:14 by percentage.

This assembly is then granulated with ACDISOL sodium croscarmellose and dry blended with magnesium stearate. The final composition is carrier/drug/polymer/excipient/lubricant in a ratio of about 60.9:11.8:11.8:15:0.5 by percentage. One gram of this final composition is compressed into an immediate release dosage form which comprises 118 mg of itraconazol.

### Example 2

Magnesium aluminometasilicate is loaded by an iterative spraying/drying process in a fluid bed granulator using a 50/50 wt % solution of itraconazol and METHOCEL E5 HPMC in DMSO with 6% solids. The solution is rapidly sprayed onto the fluidized porous particles, conservatively only loading 75% of the pores' absorbing capacity. Then the spraying is stopped while heating and fluidizing continues, allowing the solvent to evaporate leaving the drug/polymer solids behind trapped inside the pores. The process is repeated, scaling down the amount of solution applied each cycle proportional to the amount of the remaining percentage of unfilled pores. The pores will be 75% filled with drug/polymer solids after 10 iterations. Assuming 50% porosity, the final composition of the assembly is carrier/drug/polymer in a ratio of about 72:14:14 by percentage.

This assembly is then granulated with ACDISOL sodium croscarmellose and a blend of CARBOMER 71G and CARBOMER 934 available from Carbomer Inc., MA, USA, and dry blended with magnesium stearate. The final composition is carrier/drug/polymer/CARBOMER 71G/CARBOMER 934/excipient/lubricant in a ratio of about 55.4:10.8:10.8:5.0:2.5:15.0:0.5. The granules are compressed into a controlled release matrix tablet. By varying the ratios of CARBOMER 71G/CARBOMER 934 (from 7.5/0 to 0/7.5, by weight), various release duration can be achieved (from 2 hrs to 20 hrs).

### Example 3

Magnesium aluminometasilicate is loaded by an iterative spraying/drying process in a fluid bed granulator using a 75/25 wt % solution of itraconazol and METHOCEL E5 brand HPMC, in DMSO with 6% solids. The solution is rapidly sprayed onto the fluidized porous particles (magnesium aluminometasilicate), conservatively only loading 75% of the pores' absorbing capacity. Then the spraying is stopped while heating and fluidizing continues, allowing the solvent to evaporate leaving the drug/polymer solids behind trapped inside the pores. The process is repeated, scaling down the amount of solution applied each cycle proportional to the amount of the remaining percentage of unfilled pores. The pores will be 75% filled with drug/polymer solids after 10 iterations. Assuming 50% porosity, the final composition of the assembly is carrier/drug/polymer in a ratio of about 72:21:7 by weight percentage.

This assembly is then granulated with ACDISOL sodium croscarmellose and dry blended with magnesium stearate. The final composition is carrier/drug/polymer/excipient/lubricant in a ratio of about 60.9:17.7:5.9:15:0.5 by weight percentage. One gram of this final composition is compressed into an immediate release dosage form which comprises 177 mg of itraconazol.

### Example 4

Magnesium aluminometasilicate is loaded by an iterative spraying/drying process in a fluid bed granulator using a 95/5 wt % solution of itraconazol METHOCEL E5 HPMC in DMSO with 6% solids. The solution is rapidly sprayed onto the fluidized porous particles (magnesium aluminometasilicate), conservatively only loading 75% of the pores' absorbing capacity. Then the spraying is stopped while heating and fluidizing continues, allowing the solvent to evaporate leaving the drug/polymer solids behind trapped inside the pores. The process is repeated, scaling down the amount of solution applied each cycle proportional to the amount of the remaining percentage of unfilled pores. The pores will be 75% filled with drug/polymer solids after 10 iterations. Assuming 50% porosity, the final composition of the assembly is carrier/drug/polymer in a ratio of about 72:26.6:1.4 by weight percentage.

This assembly is then granulated with ACDISOL sodium croscarmellose and dry blended with magnesium stearate. The final composition is carrier/drug/polymer/excipient/lubricant in a ratio of about 60.9:22.4:1.2:15:0.5 by weight percentage. One gram of this final composition is compressed into an immediate release dosage form which comprises 224 mg of itraconazol.

### Example 5

Magnesium aluminometasilicate is loaded by an iterative spraying/drying process in a fluid bed granulator using a 50/50 wt % solution of phenytoin and METHOCEL E5 HPMC in DMSO with 6% solids. The solution is rapidly sprayed onto the fluidized porous particles (magnesium aluminometasilicate), conservatively only loading 75% of the pores' absorbing capacity. Then the spraying is stopped while heating and fluidizing continues, allowing the solvent to evaporate leaving the drug/polymer solids behind trapped inside the pores. The process is repeated, scaling down the amount of solution applied each cycle proportional to the amount of the remaining percentage of unfilled pores. The pores will be 75% filled with drug/polymer solids after 10 iterations. Assuming 50% porosity, the final composition of the assembly is carrier/drug/polymer in a ratio of about 72:14:14 by percentage.

This assembly is then granulated with ACDISOL sodium croscarmellose and dry blended with magnesium stearate. The final composition is carrier/drug/polymer/excipient/lubricant in a ratio of about 60.9:11.8:11.8:15:0.5 by percentage. One gram of this final composition is compressed into an immediate release dosage form which comprises 118 mg of phenytoin.

### Example 6

Magnesium aluminometasilicate is loaded by an iterative spraying/drying process in a fluid bed granulator using a 50/50 wt % solution of itraconazol and methacrylic acid copolymer available under the tradename EUDRAGIT L100-55 in DMSO with 6% solids. The solution is rapidly sprayed onto the fluidized porous particles (magnesium aluminometasilicate), conservatively only loading 75% of the pores' absorbing capacity. Then the spraying is stopped while heating and fluidizing continues, allowing the solvent to evaporate leaving the drug/polymer solids behind trapped inside the pores. The process is repeated, scaling down the amount of solution applied each cycle proportional to the amount of the remaining percentage of unfilled pores. The pores will be 75% filled with drug/polymer solids after 10 iterations. Assuming 50% porosity, the final composition of the assembly is carrier/drug/polymer in a ratio of about 72:14:14 by percentage.

This assembly is then granulated with ACDISOL sodium croscarmellose and dry blended with magnesium stearate. The final composition is carrier/drug/polymer/excipient/lubricant in a ratio of about 60.9:11.8:11.8:15:0.5 by percentage. One gram of this final composition is compressed into an immediate release dosage form which comprises 118 mg of itraconazol.

### Example 7

Magnesium aluminometasilicate is loaded by an iterative spraying/drying process in a fluid bed granulator using a 50/50 wt % solution of phenytoin and METHOCEL E5 HPMC in DMSO with 6% solids. The solution is rapidly sprayed onto the fluidized porous particles (magnesium aluminometasilicate), conservatively only loading 75% of the pores' absorbing capacity. Then the spraying is stopped while heating and fluidizing continues, allowing the solvent to evaporate leaving the drug/polymer solids behind trapped inside the pores. The process is repeated, scaling down the amount of solution applied each cycle proportional to the amount of the remaining percentage of unfilled pores. The pores will be 75% filled with drug/polymer solids after 10 iterations. Assuming 50% porosity, the final composition of the assembly is carrier/drug/polymer in a ratio of about 72:14:14 by percentage. This assembly is then granulated with ACDISOL sodium croscarmellose and dry blended with magnesium stearate. The final composition is carrier/drug/polymer/excipient/lubricant in a ratio of about 60.9:11.8:11.8:15:0.5 by percentage, forming porous drug-layer assembly granules.

To use the assembly with the OROS PUSH-STICK SYSTEM™, an osmotic-layer forming composition comprising, in weight percent, 58.75% sodium carboxymethyl cellulose (7H4F), 30.0% sodium chloride, 5.0% hydroxypropyl methylcellulose (METHOCEL E5), 1.0% red ferric oxide is each passed through a 40 -mesh stainless steel screen and then is blended in a GALTT fluid-bed granulator and sprayed with 5.0% hydroxypropyl cellulose (EF) solution in purified water until homogeneous granules form. These granules are passed through a 8-mesh stainless steel screen and mixed with 0.25% magnesium stearate to form an osmotic granulation.

500 mg of the porous drug-layer assembly granules from above and 250 mg of the osmotic granulation from above were compressed into bi-layer round-round tablets. The compression of these tablets are carried out with a CARVER press or a D3B MANESTY press, using a 17/64″ round punch. Next, the tablets were coated with 18 mg of a sub-coating composition comprising, in weight percent, 95% NATROSOL and 5% polyethylene glycol having a molecular weight of 3,350. Then, the sub-coated tablets were coated again with a semipermeable wall forming composition comprising cellulose acetate having an acetyl content of 39.8% and PLURONIC F68 copolymer. The wall forming composition is dissolved in acetone to make a 4% solid solution. The wall forming composition is sprayed onto the tablets in a FREUD HI-COATER coating apparatus. The membrane weight per tablet and the weight ratio of the cellulose acetate to PLURONIC F68 copolymer can be varied to obtain the target release duration. Finally, an exit orifice (155 mil) is cut mechanically on the drug-layer side of the system. The residual solvent is removed by drying the system at 30° C. and ambient humidity overnight. The system contains 59 mg of the drug.

The disclosures of each patent, patent application, and publication cited or described in this document are hereby incorporated herein by reference, in their entireties.

Each recited range includes all combinations and subcombinations of ranges, as well as specific numerals contained therein.

Various modifications of the invention, in addition to those described herein, will be apparent to those skilled in the art from the foregoing description. Such modifications are also intended to fall within the scope of the appended claims.

1. An assembly for delivering a beneficial agent with low water solubility, comprising:
a porous-particle carrier contacted with a mixture comprising the beneficial agent and a water soluble polymer.

2. The assembly of claim 1, wherein the porous-particle carrier is selected from at least one of the group consisting of magnesium aluminometasilicate, anhydrous dibasic calcium phosphate, microcrystalline cellulose, cross linked sodium carboxymethyl cellulose, soy bean hull fiber, and agglomerated silicon dioxide.

3. The assembly of claim 1, wherein the porous-particle carrier is magnesium aluminometasilicate or anhydrous dibasic calcium phosphate.

4. The assembly of claim 1, wherein the porous-particle carrier is magnesium aluminometasilicate.

5. The assembly of claim 1, wherein the porous-particle carrier is present in a range from about 20% to about 99% by weight of the assembly.

6. The assembly of claim 1, wherein the porous-particle carrier is present in a range from about 40% to about 99% by weight of the assembly.

7. The assembly of claim 1, wherein the porous-particle carrier is present in a range from about 40% to about 60% by weight of the assembly.

8. The assembly of claim 1, wherein the porous-particle carrier is present in a range from about 50% to about 99% by weight of the assembly.

9. The assembly of claim 1, wherein the porous-particle carrier is present in a range from about 60% to about 80% by weight of the assembly.

10. The assembly of claim 1, wherein the beneficial agent is selected from at least one of megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, budesnonide, progesterone, megestrol acetate, topiramate, naproxen, flurbiprofen, ketoprofen, desipramine, diclofenac, itraconazole, piroxicam, carbamazepine, phenytoin, verapamil, indinavir sulfate, lamivudine, stavudine, nelfinavir mesylate, a combination of lamivudine and zidovudine, saquinavir mesylate, ritonavir, zidovudine, didanosine, nevirapine, ganciclovir, zalcitabine, fluoexetine hydrochloride, sertraline hydrochloride, paroxetine hydrochloride, bupropion hydrochloride, nefazodone hydrochloride, mirtazpine, auroix, mianserin hydrochloride, zanamivir, olanzapine, risperidone, quetiapine fumurate, buspirone hydrochloride, alprazolam, lorazepam, leotan, clorazepate dipotassium, clozapine, sulpiride, amisulpride, methylphenidate hydrochloride, and pemoline.

11. The assembly of claim 1, wherein the beneficial agent is selected from megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, and budesnonide.

12. The assembly of claim 1, wherein the beneficial agent is present in a range from about 1% to about 60% by weight of the assembly.

13. The assembly of claim 1, wherein the beneficial agent is present in a range from about 40% to about 60% by weight of the assembly.

14. The assembly of claim 1, wherein the beneficial agent is present in a range from about 0.1 mg to about 500 mg.

15. The assembly of claim 1, wherein the beneficial agent is present in a range from about 20 mg to about 250 mg.

16. The assembly of claim 1, wherein the water soluble polymer is selected from at least one of ethyl(hydroxyethyl)cellulose, hydroxypropyl methylcellulose, hydroxyethyl cellulose modified with hydrophobic groups, and methacrylic acid copolymers.

17. The assembly of claim 1, wherein the water soluble polymer is selected from hydroxypropyl methylcellulose and methacrylic acid copolymers.

18. The assembly of claim 1, wherein the water soluble polymer is hydroxypropyl methylcellulose.

19. The assembly of claim 1, wherein the water soluble polymer is present in a range from about 1% to about 50% by weight of the assembly.

20. The assembly of claim 1, wherein the water soluble polymer is present in a range from about 10% to about 30% by weight of the assembly.

21. A method of preparing the assembly of claim 1, comprising:
providing the porous-particle carrier;
providing a solution comprising a solvent, the beneficial agent, and the water soluble polymer; and
applying the solution to the carrier.

22. The method of claim 21, wherein the solvent is selected from at least one of water, acetone, ethanol, methanol, DMSO, and methylene chloride.

23. The method of claim 21, wherein the solvent is ethanol and water.

24. The method of claim 21, wherein the solvent is ethanol and DMSO.

25. The method of claim 21, wherein the solvent is DMSO.

26. A method of delivering a beneficial agent with low water solubility to a patient, comprising:
administering the assembly of claim 1 to the patient.
